(19) United States
(12) Patent Application Publication
(10) Pub. No.: US 2021/0023343 A1
(43) Pub. Date: Jan. 28, 2021

(54) GUIDE DEVICE FOR INSERTING AT LEAST ONE SURGICAL INSTRUMENT INSIDE A BODY CAVITY

(71) Applicant: Gioachino COPPI, MODENA (IT)

(72) Inventor: Gioachino COPPI, MODENA (IT)

(21) Appl. No.: 16/490,141

(22) PCT Filed: Mar. 8, 2018
(86) PCT No.: PCT/IB2018/051522

§ 371 Date: Aug. 30, 2019

(30) Foreign Application Priority Data
Mar. 13, 2017 (IT) 102017000027573

(51) Int. Cl. A61M 25/06; A61M 25/00; A61M 25/02

(52) U.S. Cl. CPC A61M 25/0662; A61M 25/0023; A61M 2205/0266; A61M 25/008; A61M 25/02; A61M 25/0052

(57) ABSTRACT

A guide device for inserting a surgical instrument inside a body cavity comprises a guide body, a stabilisation opening and a stabilizing element which can be slidingly engaged inside of the guide body to come out from the latter through the stabilisation opening. In particular, the guide body comprises a deformable portion suitable to pass from a concave configuration, bending towards the inside to house at least partially therein the stabilizing element, to a convex configuration, bending towards the outside of the guide body restoring the cross-section of the inner lumen, and vice versa. The guide body is therefore able to shrink in diameter during the passage inside an introducer catheter and to reacquire the nominal diameter at the operative level.

The present invention relates to a guide device for inserting at least one surgical instrument inside a body cavity. This guide device is particularly suitable for inserting a guidewire at a bifurcation of a vascular cavity.

The present invention can be used in the medical surgical field, in particular for minimally invasive surgery, such as endovascular angioplasty in which the action spaces are significantly reduced and reaching the target area requires high sensitivity of manoeuvring by the surgeon.

The prior art document EP2020899B1 to Coppi describes a device for inserting a guidewire at a bifurcation of a vascular cavity. This known device comprises an elongated and flexible hollow body with a distal directing end open to allow both the passage of at least one guidewire designed to move the device itself inside the vascular cavities, and one or more endovascular surgical instruments for the execution of the surgical intervention. In order to allow a better movement of the hollow body along the predetermined feed path, especially at bifurcations and/or marked branches of the vascular cavities, the advancement of the body is assisted by the aid of a stabilizing element which, suitably inserted therein, comes out through a special lateral stabilisation opening.

Although known guide devices allow reaching the pathology to be treated, the Applicant has found that they are not however free from some drawbacks, mainly in relation to the introduction step inside the patient's body through appropriate introducer catheters.

The insertion of the guide device into a patient's body requires the use of introducer catheters. In the particular case of the known device of EP2020899B1, the body of the guide device and the stabilizing element must pass concurrently side by side in the introducer catheter, while emerging from the lateral stabilisation opening, aligned side by side. That is to say, the introducer catheter must be provided with an inner lumen capable of accommodating the body of the guide device and the stabilizing element aligned side by side.

The Applicant has therefore found that in order to be able to insert this type of known device inside the patient's body, it is necessary to use a high-diameter introducer catheter, for example 9 French (i.e. 3 mm outer diameter). This type of introducer catheter, usable for vascular access of the femoral type, is not suitable for an axillary and brachial vascular access, or for a radial access in which, since the vascular cavity is small, an insertion hole of reduced size is required.

Moreover, considering that the hole in the wall of the artery or vein and on the skin is compressed by a healthcare operator to obtain complete closure of the access, or in the more complex cases is closed by a mechanical device, the larger the size of the insertion hole for vascular access, the more complex and long is the procedure for closing this hole at the end of the angioplasty intervention, with consequent greater complications.

The object of the present invention is to provide a guide device for introducing at least one surgical instrument into a body cavity, which simplifies the introduction of the device into the patient's body, and in particular requiring use of smaller introducer catheters.

Further features and advantages will become apparent from the detailed description of a guide device for inserting at least one surgical instrument inside a body cavity, in particular a vascular cavity, as claimed below.

Further features and advantages of the present invention will become apparent from the following detailed description, illustrated by way of non-limiting example in the accompanying figures, in which:

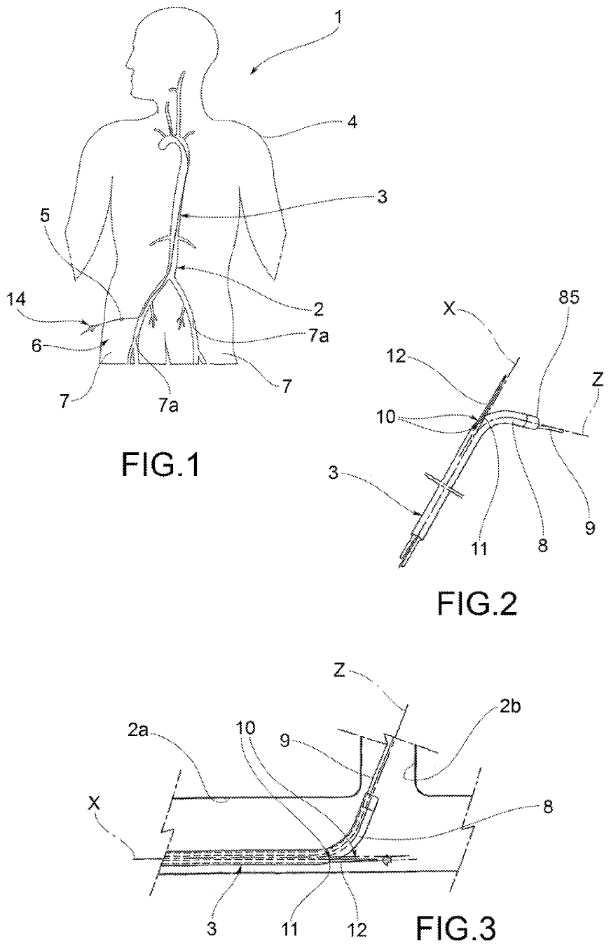

FIG. 1 is a schematic view of a guide device for inserting at least one surgical instrument inside a body cavity of a patient's body;

FIG. 2 is a broken perspective view of a detail of the device in the preceding figure;

FIG. 3 is a perspective view of the device in FIG. 2 when inserted inside a body cavity of a patient's body, in particular at a bifurcation;

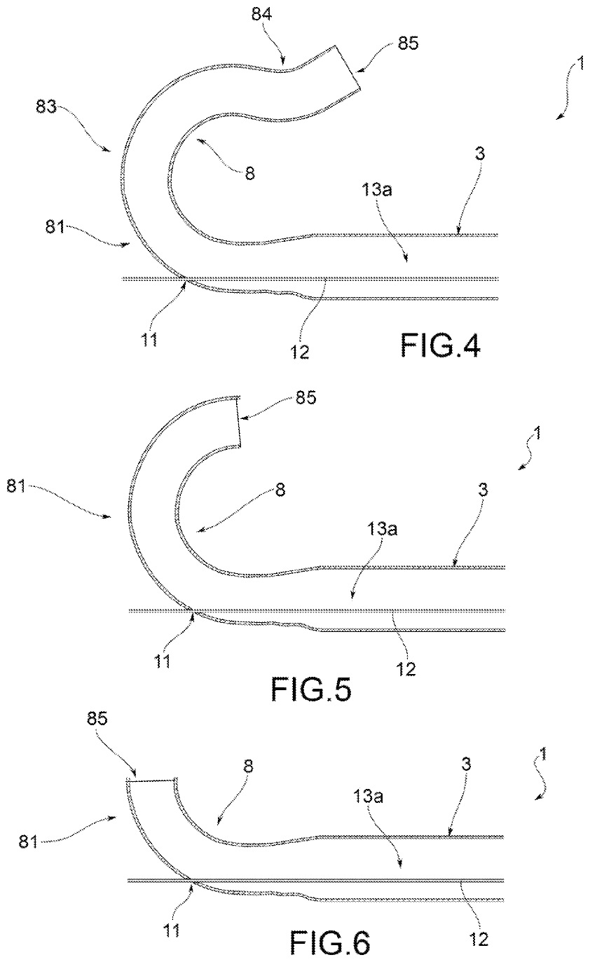

FIGS. 4 to 6 show a sectional view of some embodiments of the distal directing end of the guide device;

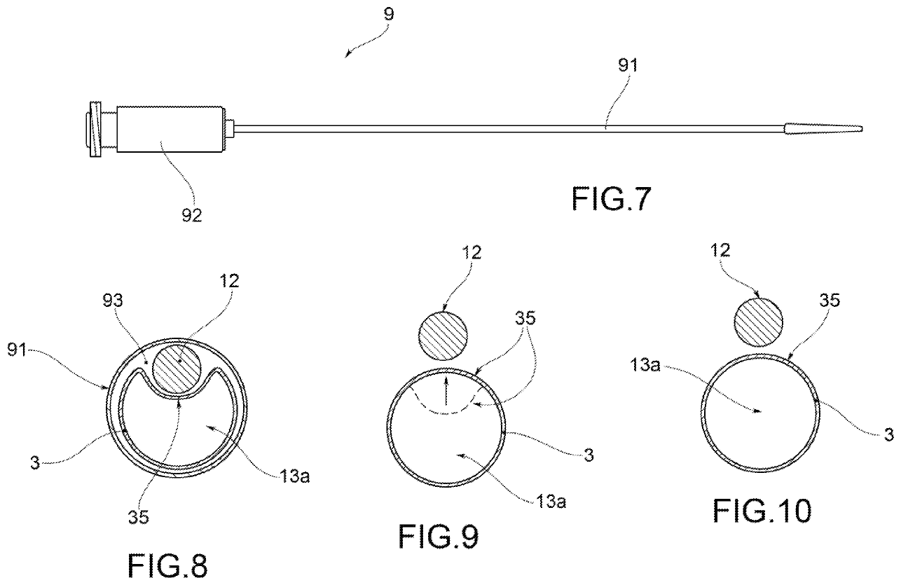

FIG. 7 is a perspective view of an introducer catheter for inserting the guide device;

FIG. 8 shows a sectional view of the distal end of the guide device when inserted inside the introducer catheter, in which the distal end and the stabilizing element are aligned side by side;

FIG. 9 shows the same section in FIG. 8, at a time point just afterwards, in which the distal end of the guide device, just come out of the introducer catheter, returns to its circular shape;

FIG. 10 shows the same section in FIG. 9, at a time point just afterwards, in which the distal end of the guide device has returned to its circular shape;

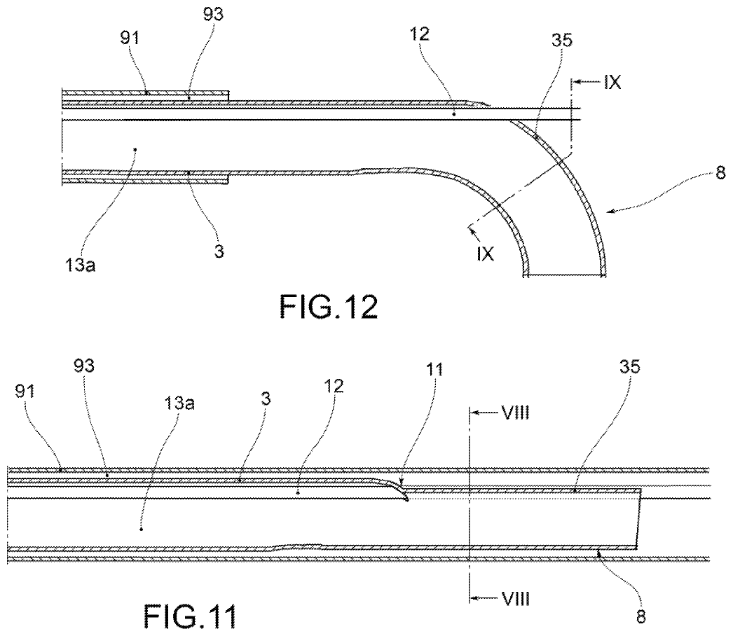

FIG. 11 shows a longitudinal sectional view of the distal end of the guide device when inserted inside the introducer catheter, corresponding to the cross section in FIG. 8;

FIG. 12 shows a longitudinal sectional view of the distal end of the guide device come out of the introducer catheter, corresponding to the cross section in FIG. 9;

FIG. 13 shows a sectional view of an embodiment example of the distal end of the guide device, in which a reinforcing armour is embedded within the thickness of the distal end wall;

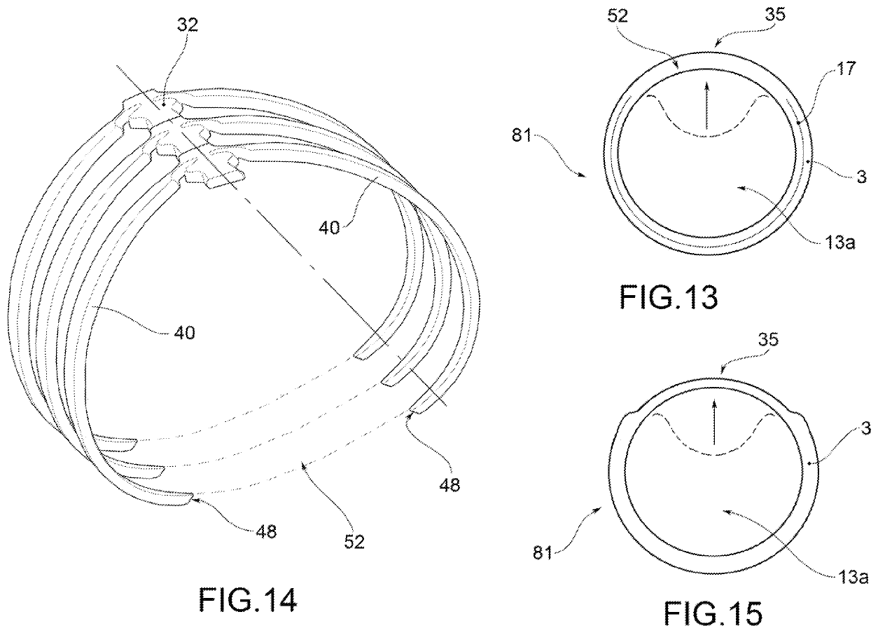

FIG. 14 shows a construction example of the reinforcing armour in FIG. 13;

FIG. 15 shows a perspective view of a further embodiment of the distal end of the guide device, in which the thickness of the distal end wall varies along the circumference;

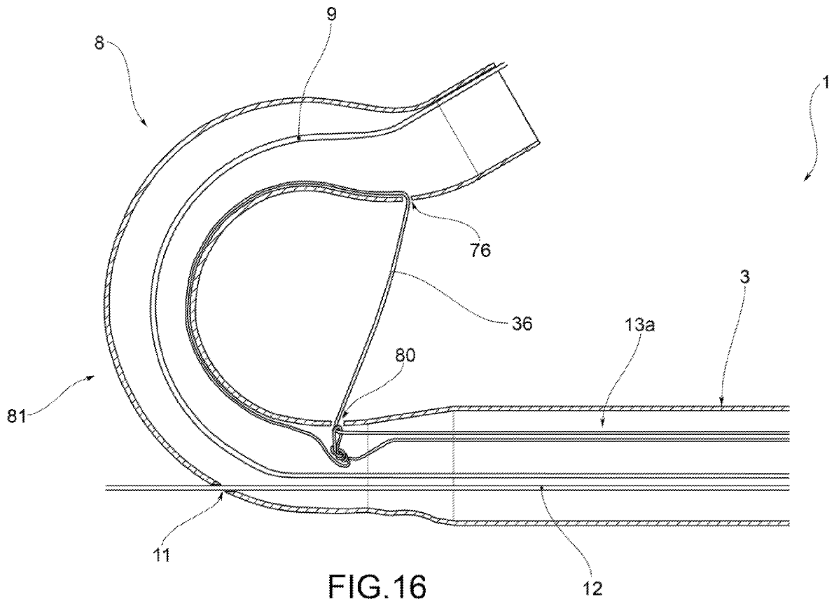

FIG. 16 shows a perspective view of the guide device provided with a pulling cable for controlling the bend of the distal end.

With reference to the aforementioned figures, reference numeral 1 indicates as a whole a guide device for inserting at least one surgical instrument (not illustrated, being known) inside one or more body cavities 2, according to the present invention.

As shown in the accompanying figures, the guide device 1 comprises at least one substantially hollow and flexible guide body 3, extending along a preferential development direction “X”.

The guide body 3 has an open directing end 8 which extends transversely to the preferential development direction “X” according to a curvilinear direction. The directing end 8 is therefore provided with a distal opening 85.

FIGS. 4 to 6 show some embodiment examples of the curvilinear direction of the directing end 8 of the guide device.

FIG. 4 shows a directing end 8 comprising a first bend 83, followed by a second bend 84. Preferably, the first bend 83 is substantially 180°, and the second bend 84 is in the opposite direction with respect to the first bend 83.

FIG. 5 shows a directing end 8 comprising only a first bend 83, substantially of 180°.

FIG. 6 shows a directing end 8 comprising only a first partial bend 83, for example substantially 45°.

The guide body 3 is provided with an inner lumen 13a, with a substantially circular cross-section, for the sliding of a guide element 9 and/or a stabilizing element 12 and/or at least a surgical instrument intended for the intervention to be carried out.

As shown in FIGS. 2 and 14, the device 1 comprises at least one guide element 9, of an elongated and flexible conformation, slidingly engageable inside the guide body itself to come out from the directing end 8, and particularly through the distal opening 85, and reach the second cavity 2b.

Advantageously, the guide device 1 comprises stabilisation means 10 associated with the guide body 3, designed to control the guide body 3 itself to improve its progress inside the cavity 2a, facilitating the positioning of the directing end 8 with respect to the second cavity 2b.

The stabilisation means 10 preferably comprise at least one stabilisation opening 11 formed through the guide body 3 and at least one stabilizing element 12 of elongated conformation (preferably over 220 cm), preferably threadlike and flexible, slidingly engageable inside the guide body 3 in which it is introduced from the outside in the proximal direction through the stabilisation opening 11 to come out from the proximal end. This insertion manoeuvre is obviously carried out when the catheter guiding body is external to the body.

Preferably, the stabilisation opening 11, i.e. the exit point of the stabilizing element, is preferably positioned at the base of the catheter bend.

The stabilizing element 12 has transversal dimensions smaller than the dimensions of the stabilisation opening 11 to easily pass through the latter.

Preferably, the stabilizing element 12 has a greater structural rigidity than the structural stiffness of the guide element 9 such that its sliding inside the guide body 3 still determines its exit through the stabilisation opening 11.

To this end, it is possible to provide that the stabilizing element 12 has the transversal dimensions of a common metal guide (0.035 inches, 0.889 mm).

The stabilisation opening 11 is preferably arranged between the directing end 8 and suitable manoeuvring means 14 (FIG. 1) located on the opposite side with respect to the directing end 8 and designed to move the guide device 1.

Advantageously, the stabilisation opening 11 is located along the guide body 3 according to a position substantially aligned with the preferential development direction “X” and substantially tangential to the curvilinear development direction of the directing end 8.

Preferably, the stabilisation opening 11 is positioned just upstream of the beginning of the first bend 83 of the guide body 3.

In use, as shown in FIG. 3, when the guide body 3 is at a marked change in direction, i.e. when the guide body must pass from a first cavity 2a to a second cavity 2b transverse to the first cavity 2a, the guide body 3 is advanced along the stabilizing element 12, which is inserted inside the guide body 3 by the proximal portion and which protrudes at least by the distal portion through the stabilisation opening 11 and is positioned so as to engage the first cavity 2a.

The stabilizing element 12 constitutes an extension of the guide body 3 along the first cavity 2a, providing the function of rotation pin of the guide body 3: the guide body 3 can thus be rotated around the stabilizing element 12 so as to allow a correct and controlled alignment of the directing end 8 with the second cavity 2b.

The sliding element 9 can thus be pushed out of the distal opening 85 of the guide body 3 when the directing end 8 is at the second cavity 2b. The guide element 9 is then inserted into the second cavity 2b travelling along at least a significant length thereof (FIG. 3), having the solid support of the guide body 3 determined by the stabilizing element 12.

As shown in FIG. 1, when the guide device 1 is used for endovascular procedures, such as angioplasty, stent applications and the like, the guide body 3 together with the stabilizing element 12 is inserted into the patient's body 4 through an introducer catheter 9, such as that shown in FIG. 7.

In particular, the guide device 1 can be inserted into the body 4 of a patient through an opening 5, formed at an area 6 identified as an entrance area. In some types of endovascular interventions, the entrance area 6 corresponds to one of the lower limbs 7 of the patient's body 4 to access, as shown in FIG. 1, a corresponding femoral artery 7a. In other cases, the entrance area 6 corresponds to one of the upper limbs, for an axillary, brachial or radial vascular access.

The introducer catheter 9 comprises a proximal portion 92, intended to remain outside the patient's body, and an introducer body 91 intended to be inserted at least partially inside a body cavity 2.

The introducer body 91 is substantially hollow and provided with a channel 93 with a substantially circular cross-section, for the sliding of the guide device 1, or of the guide body 3 and of the stabilizing element 12.

The portion of the guide body 3 extending from the stabilisation opening 11 and towards the distal end being defined as flexible tip 81, this flexible tip 81 may coincide totally or only partially with the directing end 8.

Advantageously, the guide body 3 is at least partially compressible in a transverse direction, and in particular comprises a transversely deformable and flexible portion.

Advantageously, the guide body 3 comprises, at least at the flexible tip 81, a deformable portion 35, i.e. the guide body 3 is at least partially compressible in a transverse direction.

The deformable portion 35 is adapted to take a concave configuration, that is, to fold towards the inside of the guide body 3 (reducing the section of the inner lumen 13a), to receive the stabilizing element 12, as shown in FIG. 8, which it is therefore at least partially received within the circumference of the guide body 3.

The deformable portion 35 is adapted to pass from a concave configuration to a convex configuration, or to fold outwardly of the guide body 3 (restoring the circular section of the inner lumen 13a), as shown in FIG. 9.

The deformable portion 35 is placed along the guide body 3 just after the stabilisation opening 11, on the same side as the stabilisation opening 11.

For inserting the guide device 1 inside the patient's body, the guide body 3 and the stabilizing element 12 are inserted into the channel 93 of the introducer 91.

As shown in FIG. 11, inside the introducer 91, the guide body 3 and the stabilizing element 12 are side by side. Advantageously, in order to reduce the overall section of the guide device 1, the deformable portion 35 takes a concave configuration, as shown in FIGS. 8 and 11 to accommodate the stabilizing element 12, which is partially received within the circumference of the guide body 3. Such a technical solution allows using an introducer catheter 91 of reduced size compared to known guide devices.

As soon as the guide body 3 comes out of the introducer 91, the deformable portion 35 takes, or returns to, a convex configuration, as shown in FIGS. 9 and 12, restoring the circular section of the inner lumen 13a of the guide body 3.

Such a technical solution allows using an introducer catheter 91 of reduced dimensions without the need to reduce the circular section of the guide body 3.

As can be seen in FIG. 8 and FIG. 11, the stabilizing element 12 presses against the deformable portion 35 to make it convex by reducing the section of the inner lumen 13a, and is partially received within the circumference of the guide body 3 at the directing end 8. Moreover, the overall size given by the sum of the outer diameter of the stabilizing element 12 and of the directing end 8, by the stabilisation opening 11 towards the distal end of the guide body 3, is substantially the same given by the outer diameter of the proximal part of the guide body 3, thus facilitating the passage of the guide device 1 into the introducer catheters 91.

The deformable portion 35 of the guide body 3 can be obtained in various ways.

In an embodiment example, at least the deformable portion 35 is made of a flexible polymeric material, for example of biocompatible rubber.

In a further embodiment example, shown in FIG. 15, the deformable portion 35 is obtained by thinning the wall of the guide body 3.

In yet another further embodiment example, the wall of the guide body 3 includes a reinforcing armour 17 embedded therein.

In one variant, the reinforcing armour 17 extends only partially along the circumference of the guide body 3. In this example, shown in FIG. 13, the deformable portion 35 is obtained at the portion of the part, or circumference, without reinforcing armour. The reinforcing armour 17 comprises, for example (see FIG. 14) a longitudinal element 32 which connects pairs of ribs to each other. The free ends 48 of the same pairs of ribs 40 are separate from each other and identify at least one discontinuity 52 of the same ribs 40, said discontinuity 52 being positioned at and forming the deformable portion 35.

In a different variant, the reinforcing armour 17 extends over the entire circumference of the guide body 3. In this example, the reinforcing armour 17 comprises a main portion and a flexible shape-memory portion. In this embodiment, the deformable portion 35 is obtained at the flexible portion of the reinforcing armour 17.

For example, the reinforcing armour core is made of nitinol, or steel, or a moulded polymeric material or a metal or polymer material obtained by laser moulding.

In the embodiment in FIG. 16, at least one traction cable 36 is housed in the inner lumen 13a of the guide body 3. At least a portion of the traction cable 36 runs internally through a portion of the directing end 8, following the inner bend thereof, so as to come out of a distal hole 76, formed in the wall of the directing end 8, and go back into the guide body 3 through a proximal hole 80, formed in the guide body 3, facing the distal hole 76 so as to subtend the arc formed by the directing end 8.

Preferably, the guide element 9 and the stabilizing element 12 can cooperate with the traction cable 36 in order to control with extreme precision the exact bend of the directing end 8. In fact, if on the one hand the guide element 9 and the stabilizing element 12 tend to straighten the distal end or tip of the catheter, on the other hand, by operating appropriately on the traction cable 36 it is possible to contrast said straightening and firmly maintain the exact bend imposed by the surgeon during the intervention. In any case, it will always be possible to completely release the inner lumen 13a of the guide body 3 by pulling the traction cable 36 and/or the stabilizing element 12 and/or the guide element 9 according to the specific and contingent needs of the surgeon.

Advantageously, the guide device of the present invention solves the problems encountered in the prior art and provides important advantages.

Firstly, the guide device 1 of the present invention considerably facilitates the insertion operations inside a patient's body.

In fact, in order to insert the guide device 1 it is also possible to use smaller introducer catheters, for example 5 French (or 1.67 mm outer diameter).

The guide device 1 of the present invention is suitable both for vascular access of the femoral type, and for an axial, brachial and even radial vascular access.

Moreover, being able to reduce the size of the insertion hole for vascular access, the procedure for closing such a hole at the end of the angioplasty procedure is simplified and accelerated, with consequent reduction of complications.

Moreover, the guide device 1 of the present invention facilitates alignment of the guide body 3 with respect to the transverse cavities and to the bifurcations encountered along the path towards the pathology to be treated. In particular, the presence of the stabilisation means 10 allows a rapid and correct alignment of the directing end 8, causing a considerable reduction in the intervention times.

The present invention also relates to a guide kit for inserting at least one surgical instrument inside a body cavity (2), and in particular a vascular cavity, comprising:

a guide device 1 as described above;
an introducer catheter 9, comprising an introducer body 91 substantially hollow and provided internally with a channel 93 for the sliding of the guide device 1;
wherein, when the directing end 8 and the stabilizing element 12 are placed inside the channel 93 of the introducer 91 and the stabilizing element 12 comes out at least partially through the stabilisation opening 11 of the guide body 3, the deformable portion 35 of the guide body 3 adopts the concave configuration to receive at least partially the stabilizing element 12 so that, at the directing end 8, the guide body 3 and the stabilizing element 12 run side by side and the stabilizing element 12 is partially housed inside the outer circumference of the guide body 3.

Moreover, when the directing end 8 comes out of the channel 93 of the introducer 91, the deformable portion 35 of the guide body 3 adopts the convex configuration once again, restoring the cross-section of the inner lumen 13a of the guide body 3.

The present invention also relates to a guide catheter, consisting of the guide body 3 alone, for inserting at least one surgical instrument inside a body cavity 2, and in particular a vascular cavity, said guide body 3 being:

substantially hollow and flexible extending in a preferential direction of extension X;
defining an open directing end 8, transverse to the preferential direction of extension X;
defining an inner lumen 13a for the sliding of at least one stabilizing element;
provided with at least one stabilisation opening 11 for the exit of said stabilizing element;
provided with a deformable portion 35 suitable to bend towards the inside reducing the cross-section of the inner lumen 13a when the guide body 3 is inserted inside an introducer, and to bend towards the outside of the guide body 3 restoring the cross-section of the inner lumen 13a when the guide body 3 comes out of the introducer, and vice versa.

Innovatively therefore, a guide device according to the present invention is provided with a guide body capable, due to a deformable portion, of reducing in diameter during the passage inside an introducer catheter and of reacquiring the nominal diameter at the operational level.

Of course, the guide device 1 according to the present invention lends itself to any minimally invasive surgical treatment of pathologies that also involve parts of the body other than the vascular ones, such as, for example, diseases related to the urinary, digestive, renal system and/or any other system capable of being reached by the guide device described above.

1. Guide device for inserting at least one surgical instrument inside a body cavity, and in particular a vascular cavity, comprising:
a guide body substantially hollow and flexible extending in a preferential direction of extension and defining an open directing end, transverse to the preferential direction of extension;
stabilisation means, associated with the guide body to facilitate the positioning of the directing end, comprising:
at least one stabilisation opening made through the body; at least one stabilizing element slidingly engageable inside said guide body to come out from the latter through the stabilisation opening,
wherein the guide body defines an inner lumen for the sliding of at least the stabilizing element, and wherein the guide body comprises a deformable portion suitable to pass from a concave configuration, bending towards the inside of the guide body and reducing the cross-section of the inner lumen to house at least partially therein the stabilizing element, to a convex configuration, bending towards the outside of the guide body restoring the cross-section of the inner lumen, and vice versa.

2. Guide device according to claim 1, wherein the deformable portion is placed along the guide body just after the stabilisation opening, on the same side as the stabilisation opening.

3. Guide device according to claim 1, wherein the deformable portion is made of a flexible polymeric material, for example of biocompatible rubber.

4. Guide device according to claim 1, wherein the deformable portion is made by a thinning of the wall of the guide body.

5. Guide device according to claim 1, wherein the guide body comprises, embedded in the thickness of the wall, a reinforcing armour made as plurality of pairs of ribs, and wherein the free ends of the ribs are separate from each other and identify a discontinuity positioned at and forming the deformable portion.

6. Guide device according to claim 1, wherein the guide body comprises, embedded in the thickness of the wall, a reinforcing armour composed of a main portion and a shape-memory flexible portion positioned at and forming the deformable portion.

7. Guide device according to claim 1, wherein the directing end comprises a first bend and the stabilisation opening is located just before the beginning of the first bend.

8. Guide device according to claim 1, wherein the stabilisation opening is situated in a position substantially aligned with said preferential direction of extension and substantially tangent to the curved extension direction of the directing end.

9. Guide device according to claim 1, further comprising at least one guide element, elongated and flexible, slidingly engageable inside the guide body to come out through the directing end and reach the second cavity, wherein the stabilizing element has a greater structural rigidity than the structural rigidity of the guide element.

10. Guide device according to claim 1, wherein in the inner lumen of the guide body at least a portion of a traction cable is placed, portion that runs internally through a portion of the directing end following the inner bend, comes out from a distal hole made in the directing end and goes back into the guide body through a proximal hole made in said guide body, said proximal hole facing the distal hole, so as to subtend the arch formed by the directing end.

11. Guide kit for inserting at least one surgical instrument inside a body cavity, and in particular a vascular cavity, comprising:
a guide device according to claim 1,
an introducer catheter, comprising an introducer body substantially hollow and provided internally with a channel for the sliding of the guide device;
wherein, when the directing end and the stabilizing element are placed inside the channel of the introducer and the stabilizing element comes out at least partially through the stabilisation opening of the guide body, the deformable portion of the guide body adopts the concave configuration to receive at least partially the stabilizing element so that, at the directing end, the guide body and the stabilizing element run side by side and the stabilizing element is partially housed inside the outer circumference of the guide body.

12. Guide kit according to claim 11, wherein when the directing end comes out of the channel of the introducer, the deformable portion of the guide body adopts the convex configuration once again, restoring the cross-section of the inner lumen of the guide body.

13. Guide catheter for inserting at least one surgical instrument inside a body cavity, and in particular a vascular cavity, said guide body being:
substantially hollow and flexible extending in a preferential direction of extension;
defining an open directing end, transverse to the preferential direction of extension;
defining an inner lumen for the sliding of at least one stabilizing element;
provided with at least one stabilisation opening for the exit of said stabilizing element; wherein it comprises a deformable portion suitable to bend towards the inside reducing the cross-section of the inner lumen when the guide body 3 is inserted inside an introducer, and to bend towards the outside of the guide body restoring the cross-section of the inner lumen when the guide body comes out of the introducer, and vice versa.
